(19) United States
(12) Patent Application Publication
(10) Pub. No.: US 2025/0059318 A1
(43) Pub. Date: Feb. 20, 2025

(54) BIS(2-HYDROXYETHYL) TEREPHTHALATE WITH LOW NITROGEN CONTENT

(71) Applicants: IFP ENERGIES NOUVELLES, RUEIL-MALMAISON (FR); JEPLAN, INC., Kanagawa (JP)

(72) Inventors: Guillaume BLANCKE, RUEIL-MALMAISON CEDEX (FR); David CHICHE, RUEIL-MALMAISON CEDEX (FR); Frederic FAVRE, RUEIL-MALMAISON CEDEX (FR); Damien LEINEKUGEL LE COCQ, RUEIL-MALMAISON CEDEX (FR); Charles-Philippe LIENEMANN, RUEIL-MALMAISON CEDEX (FR); Adrien MEKKI-BERRADA, RUEIL-MALMAISON CEDEX (FR)

(73) Assignees: IFP ENERGIES NOUVELLES, RUEIL-MALMAISON (FR); JEPLAN, INC., Kanagawa (JP)

(21) Appl. No.: 18/720,561

(22) PCT Filed: Dec. 6, 2022
(86) PCT No.: PCT/EP2022/084585

§ 371 Date: Jun. 14, 2024

(30) Foreign Application Priority Data
Dec. 17, 2021 (FR) FR2113821

(51) Int. Cl. C08G 63/183; C07C 69/82

(52) U.S. Cl. CPC C08G 63/183; C07C 69/82

(57) ABSTRACT

The invention relates to a composition predominantly comprising BHET and exhibiting a nitrogen content of less than or equal to 10 ppm by weight, the nitrogen content being determined according to a chemiluminescence method, with a stream of ozone at 35 cm3/min, after oxidative combustion of a sample in liquid form, prepared by diluting the composition in tetrahydrofuran, at a temperature of 1050-1100° C. and in the presence of an oxidizing mixture composed of oxygen and helium, and using a calibration curve made using a standard of diphenylamine diluted in toluene.

### TECHNICAL FIELD

The invention relates to a composition, in particular resulting from a process for the depolymerization of a polyester, more particularly from a process for the depolymerization of PET, predominantly comprising bis(2-hydroxyethyl) terephthalate (BHET) and exhibiting a low nitrogen content, in particular of less than or equal to 10 ppm by weight, said content being measured by a chemiluminescence method, said composition making it possible to produce a polyester, more particularly an r-PET or recycled PET, exhibiting little or no colouration. The invention also relates to the use of said composition to produce a polyester and to a process for the production of a colourless polyester.

### PRIOR ART

The chemical recycling of polyester, in particular of polyethylene terephthalate (PET), has formed the subject of numerous studies targeted at breaking down the polyester, recovered in the form of waste, into monomers which can then be used as feedstock of a polymerization process. The process of polymerization in particular of products resulting from the depolymerization of polyester, such as diol, diacid or diester monomers or also such as oligomers, in order to obtain PET, has also formed the subject of numerous studies.

For example, Patent U.S. Pat. No. 4,001,187 discloses processes for the production of PET, comprising a stage of continuous feeding of ethylene glycol and terephthalic acid into the esterification medium comprising bis(2-hydroxyethyl) terephthalate (BHET). The documents US 2019/0106567 and US2020031992 disclose processes for the preparation of respectively flame-retardant and dyed polyesters, by esterification of a bishydroxyalkyl terephthalate monomer with diacid mixtures, followed by polycondensation, the diacid mixtures comprising an aromatic dicarboxylic acid, preferably terephthalic acid, and respectively a carboxyphosphinic acid and a dyed aromatic dicarboxylic acid containing a sulfonate group, for example sulfoterephthalic acid. Patent Application US 2018/0340041 provides a process for the production of a polyester by polymerization, in two reaction phases, of a mixture comprising a first diol terephthalate monomer, which is predominant in the mixture, and a second monomer consisting of 2-(2-hydroxyethoxy)ethyl 2-hydroxyethyl terephthalate (BHET-deg), which is in the minority in the mixture, the first esterification phase being carried out at a moderate temperature.

In the same way, the document MX 2007/004429 discloses the production of a polyester, comprising the depolymerization by glycolysis of PET flakes at atmospheric pressure in the presence of ethylene glycol in a bis(2-hydroxyethyl) terephthalate (BHET) base. The intermediate product obtained on conclusion of the depolymerization step is filtered through a sintered filter in order to retain particles of at least 25 μm before being introduced into the polymerization reactor. Patent Application WO 2017/006217 discloses the process for the preparation of a glycol-modified polyethylene terephthalate (r-GPET) comprising a step of depolymerization of a PET in the presence of a mixture of monoethylene glycol (MEG) and neopentyl glycol, followed directly by a step of polymerization of the reaction effluent. Patent Application FR 3053691 describes, for its part, a process for the depolymerization of a polyester feedstock, comprising in particular from 0.1% to 10% by weight of pigments, by glycolysis in the presence of ethylene glycol. A bis(2-hydroxyethyl) terephthalate (BHET) monomer effluent, obtained after specific separation and purification steps, can feed a polymerization step for the purpose of producing PET. Patent JP3715812 describes the production of refined BHET from PET, it being possible for the BHET obtained to be used as starting material in a process for the production of plastic products. Patent EP 1 120 394 discloses the optional use of high-purity bis(2-hydroxyethyl) terephthalate (BHET) as starting material for the production of a polyester, the BHET being obtained by depolymerization of a polyester.

While they disclose the polymerization of monomeric products, in particular of BHET, which result from the depolymerization of PET by glycolysis, the documents cited do not, however, give any information on the quality of the intermediate products resulting from the depolymerization of PET, in order to make it possible to obtain an r-PET (that is to say a recycled PET and more particularly one obtained after a chemical recycling of polyester materials) of good quality and in particular which is low in colour, indeed even colourless.

In point of fact, depending on the applications targeted, several types of additives can be introduced into the final polyester. These are in particular organic molecules comprising one or more nitrogen atoms, such as the light stabilizers described by Li et al. (Bo Li, Study of the Migration of Stabilizer and Plasticizer from Polyethylene Terephthalate into Food Simulants, Journal of Chromatographic Science, 2016, Vol. 54, No. 6, 939-951), chain extenders or flame retardants (Swoboda Benjamin Swoboda, Amélioration du comportement au feu de PET et d′alliages PET/PC recyclés [Improvement in the Fire Behaviour of Recycled PET and PET/PC Alloys], Materials Chemistry, University doctoral dissertation, Montpellier: University of Montpellier II, 2007, 280 p.), the anthranilamide acetaldehyde scavenger as described in Patent U.S. Pat. No. 6,274,212. Furthermore, to enhance the oxygen barrier properties, the polyester used in the manufacture of containers for food use may be combined with other polymers such as nylon MXD6, a generic name given to a range of crystalline polyamides. This combination also introduces the presence of molecules containing nitrogen atoms.

It is known that the additives introduced into the polymeric formulations according to their intended applications may be detrimental and affect the type of recycling to be envisaged for recycling the plastics materials. The reason is that these additives may affect the mechanical properties of the recycled polymers, preventing mechanical recycling, or else may affect the colouring of the polymers, especially of polyesters recycled chemically, in particular by depolymerization and subsequent repolymerization of the monomers obtained.

The colour of the resulting polyester may be characterized by a colorimetry method as described in ASTM D6290 2019. The measurements are expressed in the CIE L*a*b* reference system. The parameter L′ represents the lightness (or luminance), which increases when it approaches 100. The values of the parameter a* correspond to a colour ranging from green (negative values) to red (positive values). The values of the parameter b*, lastly, correspond to a colour varying from blue (negative values) to yellow (positive values). Patent U.S. Pat. No. 8,431,202 discloses that the desirable colour for a polyester is indicated generally by a value for coordinate a* of preferably from minus 4.4 (−4.4) to plus 1.6 (+1.6) and a value for coordinates b* of preferably from minus 8.6 (−8.6) to plus 10.2 (+10.2).

The Applicant Company's inventors have found, then, that the quality of BHET, a monomer of PET, and more particularly the quality of the r-BHET obtained from the depolymerization of a PET feedstock, and more especially its nitrogen content, greatly affects the quality, especially the colouring, of the r-PET prepared.

### SUMMARY OF THE INVENTION

The invention provides a composition predominantly comprising BHET and exhibiting a nitrogen content of less than or equal to 10 ppm by weight, the nitrogen content being determined according to a chemiluminescence method, with a stream of ozone at 35 cm³/min, after oxidative combustion of a sample in liquid form, prepared by diluting the composition in tetrahydrofuran, at a temperature of 1050-1100° C. and in the presence of an oxidizing mixture composed of oxygen and helium, and using a calibration curve made using a standard of diphenylamine diluted in toluene.

The advantage of the present invention lies in the capacity of the BHET-based composition to produce, after polymerization, a polyester, in particular a PET, which is low in colour or even colourless. The clear polyester thus obtained may be used in a wide variety of applications-applications in which the polyester base material will be highly coloured, for which the polyester will be integrated into highly coloured formulations, and not only grey formulations, through to applications of the bottle type, for example, for which the material may be transparent and colourless or with a slight blue tint.

Another advantage of the present invention lies in the origin of the BHET-based composition, which originates advantageously from networks for the recycling of plastics, set up in recent years by national and international organisations in order to combat plastic pollution. The reason is that the BHET-based composition of the present invention is very advantageously obtained at the outcome of processes for depolymerization by glycolysis of polyester, such as PET, in the presence of diol. The BHET obtained from these depolymerization processes is then termed r-BHET and the PET prepared by polymerization from r-BHET is termed r-PET (as opposed to the virgin PET or resin obtained from the direct polymerization of fresh terephthalic acid and fresh ethylene glycol). The present invention therefore plays a part in the combating of plastic pollution.

The present invention thus also relates to the use of said composition to prepare a polyester, preferably a PET. It also provides a process for the production of a polyester, comprising: a) a step of esterification of a feedstock comprising at least the composition according to any of claims 1 to 4, and optionally at least one dicarboxylic acid, preferably selected from terephthalic acid and isophthalic acid, and/or at least one diol, preferably selected from ethylene glycol, diethylene glycol, butylene glycol, cyclohexanedimethanol, neopentyl glycol or mixtures thereof, the preferred diol being ethylene glycol; then b) a step of polycondensation.

### DESCRIPTION OF THE EMBODIMENTS

According to the invention, the terms “bis(2-hydroxyethyl) terephthalate” and “BHET” denote the same compound and are interchangeable. Similarly, the terms “bis(2-hydroxyethyl) isophthalate” and “BHEI” denote the same compound and are interchangeable. The terms “2-(2-hydroxyethoxy)ethyl 2-hydroxyethyl terephthalate” and “BHET-deg” denote the same compound and are interchangeable too.

According to the invention, the term “polyester” denotes a thermoplastic polymer, advantageously a saturated polymer (as opposed to thermosetting polyesters) whose elementary repeating units are diol diesters, and more particularly at least alkylene terephthalate units. The polymer chain may also comprise alkylene isophthalates and/or dialkyl terephthalate units. Thus, according to the invention, the term “polyester” is used to denote a poly(alkylene terephthalate) (or polyalkylene terephthalate, according to an alternative terminology). The polyester according to the invention may, for example, be poly(ethylene terephthalate) (or polyethylene terephthalate, PET), poly(butylene terephthalate) (or polybutylene terephthalate, PBT) or poly(trimethylene terephthalate) (or polytrimethylene terephthalate, PTT). The polyester according to the invention can also comprise other units on its main polymer chain, such as vinyl or polyol units, according to the final properties desired for the polymer and according to the applications targeted. According to the invention, the preferred polyester is polyethylene terephthalate or poly(ethylene terephthalate), also called simply PET.

According to the invention, the terms “diol” and “glycol” are used without distinction and correspond to compounds comprising 2 hydroxyl-OH groups and preferably comprising between 2 and 12 carbon atoms, preferentially between 2 and 4 carbon atoms. The preferred diol is ethylene glycol, also referred to as monoethylene glycol or MEG.

According to the present invention, the expressions “of between . . . and . . . ” and “between . . . and . . . ” are equivalent and mean that the limiting values of the interval are included in the range of values which are described. If such is not the case and if the limiting values are not included in the range described, such information will be introduced by the present invention.

Within the meaning of the present invention, the various ranges of parameters for a given stage, such as the ranges of pressures and the ranges of temperatures, can be used alone or in combination. For example, within the meaning of the present invention, a range of preferred pressure values can be combined with a range of more preferred temperature values.

In the continuation of the text, specific embodiments of the invention may be described. They can be implemented separately or combined together, without limitation of combinations when this is technically feasible.

According to the invention, the pressures are absolute pressures and are given in MPa.

The invention therefore relates to a composition predominantly comprising BHET, in particular at least 90% by weight of BHET, preferably at least 95% by weight of BHET and more preferably at least 98% by weight of BHET, and exhibiting a nitrogen content of less than or equal to 10 ppm by weight, preferably less than or equal to 5 ppm by weight and very advantageously less than 3 ppm by weight. The nitrogen content is advantageously determined by a chemiluminescence method using, in particular, a PAC-Antek Multitek VNS instrument, especially one equipped with a vertical oven, syringe-mode injection and a CTC Analytics PAL System 748 sample changer. More particularly, the nitrogen content is determined by chemiluminescence with a stream of ozone at preferably 35 cm³/min, after oxidative combustion of a sample in liquid form, advantageously prepared by diluting the composition in tetrahydrofuran (THF) in particular by 10-fold dilution, preferably 10 μl or 20 μl of said sample in liquid form advantageously contained in a quartz tube, at a temperature of 1050-1100° C. and in the presence of an oxidizing mixture composed of oxygen and helium (preferably in a stream of oxygen at 450 cm³/min and helium at 130 cm³/min), and using a calibration curve made using a standard of diphenylamine diluted in toluene.

More generally, the principle of analysis by chemiluminescence lies in the decomposition of a sample comprising nitrogen, in liquid form, heated at a high temperature, of 1050-1100° C. for example. Said sample undergoes oxidative combustion in the presence of oxygen. The products resulting from this combustion are CO₂, H₂O, nitrogen oxides NOx, and SO₂. The nitrogen compounds obtained are then detected by chemiluminescence, based on the fact that NO reacts with the ozone to produce nitrogen dioxide in an excited state (NO₂*), according to the following reactions:

NO+O₃→NO₂*+O₂, and

NO₂*→NO₂+photon·(hv)

The light emitted by the return of NO₂* to its ground state is detected by a photomultiplier. The electrical signal obtained is a function of the amount of nitrogen present in the test sample. The intensity of the signals obtained is related to the nitrogen concentration using a calibration curve made beforehand using a standard, such as diphenylamine, diluted in a solvent, such as toluene, to give different total nitrogen contents.

The composition based on bis(2-hydroxyethyl) terephthalate (BHET) is preferably obtained by a process of depolymerizing a polyester feedstock, preferably comprising PET. For example, the BHET-based composition is the product obtained at the outcome of the depolymerization process described in Patent FR 3053691. The polyester feedstock of the targeted depolymerization processes comes advantageously from collecting and sorting channels which are in turn integrated into a system for recycling of plastics in particular. Said polyester feedstock therefore comprises, in particular, post-consumer products and/or wastes that are based on polyester, more particularly on PET. The product obtained at the outcome of the processes for treating such polyester feedstocks, which comprise a step of depolymerization, preferably by glycolysis in the presence of diol, preferably ethylene glycol, or by methanolysis in the presence of methanol, typically comprises predominantly BHET and may be termed r-BHET. Very advantageously, the composition according to the invention is one of these products, obtained at the outcome of processes for treating such polyester feedstocks, preferably comprising PET, which comprise a step of depolymerization, preferably by glycolysis in the presence of diol, preferably ethylene glycol, or by methanolysis in the presence of methanol, more preferably by glycolysis in the presence of ethylene glycol.

According to one particular embodiment of the invention, the BHET-based composition according to the invention may further comprise bis(2-hydroxyethyl) isophthalate (BHEI), preferably in a molar amount such that the molar ratio (BHEI/[BHET+BHEI]) of the number of moles of BHEI to the number of moles of both BHET and BHEI that are present in the composition is less than or equal to 10.0 mol %, preferably less than or equal to 5.0 mol %, more preferably less than or equal to 1.0 mol % and very preferably less than or equal to 0.5 mol %. Moreover, if the composition comprises BHEI, the molar ratio (BHEI/[BHET+BHEI]) is greater than or equal to 0.001 mol %, preferably greater than or equal to 0.01 mol %, more preferably greater than or equal to 0.05 mol %.

According to another particular embodiment of the invention, the BHET-based composition according to the invention may further comprise 2-(2-hydroxyethoxy)ethyl 2-hydroxyethyl terephthalate (BHET-deg), preferably in a molar amount such that the molar ratio (BHET-deg/[BHET+BHET-deg]) of the number of moles of BHET-deg to the number of moles of both BHET and BHET-deg that are present in the composition is less than or equal to 10.0 mol %, preferably less than or equal to 5.0 mol %, more preferably less than or equal to 1.0 mol %. Moreover, if the composition comprises BHET-deg, the molar ratio (BHET-deg/[BHET+BHET-deg]) is greater than or equal to 0.001 mol %, preferably greater than or equal to 0.05 mol %, more preferably greater than or equal to 0.10 mol %, very preferably greater than or equal to 0.50 mol %.

One or the other of these two particular embodiments of the invention may optionally happen to be the case or cases of products obtained at the outcome of processes for treating polyester feedstocks that comprise a step of depolymerization, preferably by glycolysis in the presence of diol, preferably ethylene glycol, or by methanolysis in the presence of methanol, more preferably by glycolysis in the presence of ethylene glycol.

A composition according to the invention thus very advantageously enables the production, after polymerization, of a polyester, preferably a PET, and more particularly an r-PET having a clear or even colourless colouration.

Hence the invention also relates to the use of the composition according to the invention, optionally mixed with at least one dicarboxylic acid, preferably selected from terephthalic acid and isophthalic acid, and/or at least one diol, preferably selected from ethylene glycol, diethylene glycol, butylene glycol, cyclohexanedimethanol, neopentyl glycol or mixtures thereof, the preferred diol being ethylene glycol, to prepare a polyester, preferably a PET.

The invention thus also relates to a process for the production of a polyester, comprising, preferably consisting of:
a) a step of esterification of a feedstock comprising at least the composition according to the invention, and optionally at least one dicarboxylic acid, preferably selected from terephthalic acid and isophthalic acid, and/or at least one diol, preferably selected from ethylene glycol, diethylene glycol, butylene glycol, cyclohexanedimethanol, neopentyl glycol or mixtures thereof, the preferred diol being ethylene glycol; then
b) a step of polycondensation.

Advantageously, step a) is implemented at a temperature of between 15° and 350° C., preferably between 20° and 300° C., more preferably between 25° and 285° C. Preferably, step a) is implemented at a pressure between 0.05 and 1.0 MPa, preferably between 0.1 and 0.5 MPa. Very advantageously, step a) is implemented with a residence time between 0.5 and 10.0 hours, preferably between 1.0 and 6.0 hours, the residence time being defined here as the ratio of the reaction volume of a reactor implemented in step a) to the volume flow rate of the liquid stream emerging from said reactor.

A polymerization catalyst, preferably based on antimony, titanium, germanium, aluminium, zinc acetate, calcium acetate and/or manganese acetate, can optionally be introduced in step a).

The reaction carried out in step a) generates a diol compound which is advantageously separated during step a), for example by withdrawing, distillation and/or adsorption. Water may also be formed. The water then formed is itself also advantageously separated during step a).

The process for the production of a polyester according to the invention advantageously comprises a step b) of polycondensation at the end of step a). Step b) may advantageously implement one or more, preferably one or two, polycondensation substeps, for example at least one, preferably one substep of polycondensation in liquid or molten phase, optionally followed by at least one, preferably one substep of polycondensation in solid phase.

Very advantageously, the polycondensation step b) implements at least one polymerization section, preferably one or two polymerization sections, advantageously operated in the liquid or melt phase, said polymerization section(s) being implemented at a temperature greater than the temperature at which step a) is implemented, preferably at a temperature of between 19° and 400° C., preferentially between 22° and 350° C., in a preferred way between 265 and 300° C., preferably at a pressure between 0.01 and 100.00 kPa, preferentially between 0.05 and 10.00 kPa, and in a preferred way with a residence time of between 0.1 and 5.0 hours, preferably between 0.5 and 4 hours, preferentially between 1.0 and 3.0 hours. According to the invention, the residence time in the polymerization section of step b) is defined as the ratio of the reaction volume of a reactor implemented in said polymerization section to the volume flow rate of the liquid stream, comprising the polyester produced, emerging from said reactor.

The polymerization reaction may optionally be continued in a polycondensation section situated downstream of the polymerization section and operated in solid phase, preferably at a temperature (especially a product temperature) of between 19° and 250° C., more preferably between 20° and 230° C. This operation is conducted in continuous mode or in batch mode accordingly. The polycondensation section may preferably be operated under an inert atmosphere, for example in a stream of nitrogen at a pressure close to atmospheric pressure, or under vacuum (especially at a pressure of between 0.01 and 100 kPa, or even between 0.01 et 10 kPa). The residence time (defined as the time during which the product is subjected to the polycondensation conditions in said polycondensation section) is of between 5 and 20 hours, preferably between 10 and 16 hours. Said polycondensation section can advantageously be preceded by a crystallization section, thus located between the polymerization section and the polycondensation section, in which the polyester formed, obtained at the end of the polymerization section, is advantageously crystallized, it being possible for said crystallization section to be operated at a temperature preferably between 11° and 210° C. and for a residence time (defined as the time during which the product is subjected to the crystallization conditions in said section) preferably between 0.5 and 6 hours.

Step b) is preferably carried out in the presence of a polymerization catalyst, in particular based on antimony, titanium, germanium, aluminium, zinc acetate, calcium acetate and/or manganese acetate.

Additives can be introduced in the polycondensation step b). The additives optionally introduced in step b) may be, for example, as follows: agents which inhibit secondary etherification reactions, such as, for example, amines (n-butylamine, diisopropylamine or triethylamine), sodium hydroxide or organic hydroxides or lithium carbonate, stabilizing agents, such as phosphites or phosphates, and compounds of polyamide type for reducing the amount of decomposition product, such as acetaldehyde.

Via the process according to the invention, advantageously implementing the polymerization of the BHET-based composition according to the invention with a nitrogen content, determined by the method of chemiluminescence set out above, of less than or equal to 10 ppm by weight, preferably less than or equal to 5 ppm by weight, very advantageously less than 3 ppm by weight, it is possible to produce a polyester, preferably a PET, which has little colouration or is even colourless. Very advantageously, it is possible via the present invention to produce, by polymerization of the BHET-based composition according to the invention, a polyester, preferably a PET, having a value of coordinates b*, determined by colorimetry as described in ASTM D6290 2019, of advantageously between −10.0 and +10.0, preferably between −8.0 and +8.0.

Patent U.S. Pat. No. 8,431,202 states that the addition of cobalt additives or of organic toners (alternatively called colour correctors) allows the yellow colour of the resin, measured in terms of b*, to be minimized or eliminated. Hence the process according to the invention, implementing the polymerization of a BHET-based composition exhibiting a nitrogen content as measured by chemiluminescence of less than or equal to 10 ppm by weight, preferably less than or equal to 5 ppm by weight, very advantageously less than 3 ppm by weight, makes it possible to reduce the proportion of colour correctors needed in light of the intended application of the polyester.

The following examples illustrate the invention without limiting the scope thereof.

### EXAMPLES

### Example 1

A total nitrogen content of 14.0 ppm by weight is measured in a first batch of BHET, obtained by glycolytic depolymerization of a PET feedstock.

The nitrogen content is determined by a chemiluminescence method, using a PAC-Antek Multitek VNS instrument, equipped with a vertical oven, syringe-mode injection and a CTC Analytics PAL System 748 sample changer, and by:
introduction of 10 μl of sample in liquid form (prepared by 10-fold dilution in THF of the first batch of BHET) in a quartz tube heated to 1050° C.,
oxidative combustion of the sample in the presence of an oxidizing stream composed of helium (130 cm³/min) and oxygen (450 cm³/min), producing CO₂, H₂O, NOx, and SO₂,
detection by a photomultiplier of the light emitted following the reaction of the resultant NO obtained with a stream of ozone (35 cm³/min), producing nitrogen dioxide in an excited state (NO₂*), followed by the return of NO₂* to its ground state,
determination of the amount of nitrogen by comparing the intensity of the electrical signal obtained with a calibration curve made beforehand using a standard of diphenylamine diluted in toluene.

This BHET is entered into an esterification step a) carried out at 275° C. under 0.15 MPa for 90 minutes, in the presence of 250 ppm of Sb₂O₃ catalyst.

The reaction medium is subsequently subjected, in a first polycondensation step, to a temperature of 285° C. and a pressure of 0.1 kPa for 105 min.

Subsequently, after a prior step of crystallization for 2 h at 125° C. (that is to say, at the temperature of the granules), the preceding polyester obtained at the end of the first polycondensation step is entered into a solid-phase polycondensation step at 200° C., at atmospheric pressure under nitrogen circulation.

The PET obtained at the end of polycondensation is coloured, with a pronounced yellow-coloured appearance.

The mean of 10 colorimetric measurements made according to ASTM D6290 2019 in reflectance gives the following value for the coordinate b*: b*=18.4.

### Example 2

A second batch of BHET is obtained by a process for depolymerization of a PET feedstock in the presence of ethylene glycol. The total nitrogen content measured on this second batch of BET by the chemiluminescence method described above in Example 1 is less than 2.3 ppm by weight.

This BHET is entered into an esterification step a) carried out at 275° C. under 0.15 MPa for 93 minutes, in the presence of 250 ppm of Sb₂O₃ catalyst.

The reaction medium is subsequently subjected, in a first polycondensation step, to a temperature of 285° C. and a pressure of 0.1 kPa for 64 min.

Subsequently, after a preliminary stage of crystallization for 2 h at 125° C. (that is to say, at the temperature of the granules), the preceding polyester obtained at the end of the first polycondensation stage is involved in a solid-phase polycondensation stage at 200° C., at atmospheric pressure under nitrogen circulation.

The PET obtained at the end of polycondensation has little colouration and has a less coloured appearance than the PET obtained by the process described in Example 1.

The mean of 10 colorimetric measurements made according to ASTM D6290 2019 in reflectance gives the following value for the coordinate b*: b*=7.37.

### Example 3

A further batch of BHET is obtained by a process for depolymerization of a PET feedstock in the presence of ethylene glycol. The total nitrogen content measured on this batch of BET by the chemiluminescence method described above in Example 1 is less than 2.3 ppm by weight.

This BHET is entered into an esterification step a) carried out at 275° C. under 0.15 MPa for 90 minutes, in the presence of 250 ppm of Sb₂O₃ catalyst.

The reaction medium is subsequently subjected, in a first polycondensation step, to a temperature of 285° C. and a pressure of 0.1 kPa for 69 min.

Subsequently, after a preliminary stage of crystallization for 2 h at 125° C. (that is to say, at the temperature of the granules), the preceding polyester obtained at the end of the first polycondensation stage is involved in a solid-phase polycondensation stage at 200° C., at atmospheric pressure under nitrogen circulation.

The PET obtained at the end of polycondensation has little colouration and has a less coloured appearance than the PET obtained by the process described in Example 1.

The mean of 10 colorimetric measurements made according to ASTM D6290 2019 in reflectance gives the following value for the coordinate b*: b*=6.74.

1. Composition predominantly comprising bis(2-hydroxyethyl) terephthalate, also called BHET, and exhibiting a nitrogen content of less than or equal to 10 ppm by weight, the nitrogen content being determined according to a chemiluminescence method, with a stream of ozone at 35 cm³/min, after oxidative combustion of a sample in liquid form, prepared by diluting the composition in tetrahydrofuran, at a temperature of 1050-1100° C. and in the presence of an oxidizing mixture composed of oxygen and helium, and using a calibration curve made using a standard of diphenylamine diluted in toluene.

2. Composition according to claim 1, wherein the nitrogen content is less than or equal to 5 ppm by weight, preferably less than or equal to 3 ppm by weight.

3. Composition according to claim 1, obtained by a process of treatment of a polyester feedstock, preferably comprising PET, which comprises a step of depolymerization by glycolysis in the presence of ethylene glycol or by methanolysis in the presence of methanol.

4. Composition according to claim 1, comprising at least 90% by weight of BHET, preferably at least 95% by weight of BHET and more preferably at least 98% by weight of BHET.

5. Composition according to claim 1, comprising bis(2-hydroxyethyl) isophthalate, also called BHEI, preferably in a molar ratio (BHEI/[BHET+BHEI]) of the number of moles of BHEI to the number of moles of both BHET and BHEI that are present in the composition of less than or equal to 10.0%, preferably less than or equal to 5.0%, more preferably less than or equal to 1.0% and very preferably less than or equal to 0.5%.

6. Composition according to claim 1, comprising 2-(2-hydroxyethoxy)ethyl 2-hydroxyethyl terephthalate, also called BHET-deg, preferably in a molar ratio (BHET-deg/[BHET+BHET (deg)]) of the number of moles of BHET-deg to the number of moles of both BHET and BHET-deg that are present in the composition of less than or equal to 10.0 mol %, preferably less than or equal to 5.0 mol %, more preferably less than or equal to 1.0 mol %.

7. A process according to claim 9, wherein PET is prepared.

8. A process according to claim 7, wherein the composition according to any of claims 1 to 6 is mixed with at least one dicarboxylic acid, preferably selected from terephthalic acid and isophthalic acid, and/or at least one diol, preferably selected from ethylene glycol, diethylene glycol, butylene glycol, cyclohexanedimethanol, neopentyl glycol or mixtures thereof, the preferred diol being ethylene glycol.

9. Process for the production of a polyester, comprising:
a) a step of esterification of a feedstock comprising at least the composition according to claim 1, and optionally at least one dicarboxylic acid, preferably selected from terephthalic acid and isophthalic acid, and/or at least one diol, preferably selected from ethylene glycol, diethylene glycol, butylene glycol, cyclohexanedimethanol, neopentyl glycol or mixtures thereof, the preferred diol being ethylene glycol; then
b) a step of polycondensation.

10. Process according to claim 1, wherein step a) is implemented at a temperature of between 15° and 350° C., preferably between 20° and 300° C., more preferably between 25° and 285° C., and preferably at a pressure of between 0.05 and 1.0 MPa, more preferably between 0.1 and 0.5 MPa.

11. Process according to claim 9, wherein step b) implements one or more polycondensation substeps, for example at least one polycondensation substep in liquid or molten phase, followed optionally by at least one polycondensation substep in solid phase.

12. Process according to claim 9, wherein step b) implements at least one polymerization section, advantageously operated in liquid or molten phase, and implemented at a temperature greater than the temperature at which step a) is implemented, preferably at a temperature of between 19° and 400° C., more preferably between 22° and 350° C., very preferably between 265 and 300° C., and preferably at a pressure of between 0.01 and 100.0 kPa, more preferably between 0.05 and 10.00 kPa.

13. Process according to claim 9, wherein step b) is implemented in the presence of a polymerization catalyst, preferably based on antimony, titanium, germanium, aluminium, zinc acetate, calcium acetate and/or manganese acetate.
